(19) United States
(12) Patent Application Publication
(10) Pub. No.: US 2003/0004165 A1
(43) Pub. Date: Jan. 2, 2003

(54) Polyazanaphthalene compounds and pharmaceutical use thereof

(75) Inventors: Yukio Iino, Kawasaki-shi (JP); Toshihiro Hatanaka, Kawasaki-shi (JP); Yuko Satake, Kawasaki-shi (JP); Kenji Takehana, Kawasaki-shi (JP); Akiko Oonuki, Kawasaki-shi (JP); Tsuyoshi Kobayashi, Kawasaki-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: AJINOMOTO CO. INC, Tokyo (JP)

(21) Appl. No.: 10/136,359

(22) Filed: May 2, 2002

Related U.S. Application Data
(63) Continuation of application No. PCT/JP00/07696, filed on Nov. 1, 2000.

(30) Foreign Application Priority Data
Nov. 2, 1999 (JP) 11-311773

(51) Int. Cl. C07D 257/08; C07D 257/12; C07D 487/02; A61K 31/53; A61K 31/519; A61K 31/503

(52) U.S. Cl. 514/243; 514/251; 514/262.1; 514/264.1; 514/248; 514/249; 544/179; 544/184; 544/236; 544/256; 544/257; 544/279

(57) ABSTRACT

The present invention relates to an inhibitor of matrix metalloprotease (MMP) production and tumor necrosis factor (TNF) production, and medicines for the treatment of diseases such as chronic rheumatoid arthritis, osteoarthritis, allergic diseases, psoriasis, transplant rejection, arterial sclerosis, ischemic re-perfusion disorder, diabetic nephritic and ocular diseases, cancer, autoimmune glomerulonephritis, infectious diseases, Crohn's disease, inflammatory intestinal diseases and autoimmune hepatitis, each comprising certain polyazanaphthalene compounds or pharmaceutically acceptable salts thereof as active ingredients.

### BACKGROUND OF THE INVENTION

The present invention relates to the medicine useful for the treatment of the diseases such as chronic rheumatoid arthritis, osteoarthritis, and cancer, which produce excess tumor necrosis factors (TNF) and matrix metalloprotease (MMP) that is one of tissue degrading enzymes.

In such diseases like chronic rheumatoid arthritis and osteoarthritis, the joint destruction develops by the activity of tissue degrading enzymes such as MMP expressed and induced in local joints for certain causes, and it has become major concerns that quality of life of patients is seriously impaired. Existing medicine can remove the pain caused by inflammation to a certain degree by the symptomatic treatment, though it can not prevent the development of the joint destruction. Therefore, the medicine that can prevent the development of the joint destruction is expected.

Under such situations, though enzyme inhibitors of MMP have been studied, they have not been practically used. This is because it is difficult to prevent the tissue degradation completely by a unique enzyme inhibitor as various MMP sub-type relate to local inflammation.

On the other hand, MMP production inhibitor can prevent the activities of various MMP at the same time because it works on producing cells of them and is expected to have higher effectiveness of preventing tissues from being degraded than the aforementioned enzyme inhibitors.

Besides, inflammatory cytokines such as TNF, activate the cells producing tissue degrading enzymes in local inflammation of articular synovial tissues and work as an inducer of those tissue degrading enzymes. Therefore, it is contemplated that inhibiting the function of TNF can prevent the following inflamation, and such inhibitors are being developed widely. While biomolecules such as an anti-TNF antibody and a soluble receptor are being admitted to be effective, effective compounds as inhibitors of low molecules have not yet been found, and compounds of low molecules that can effectively prevent TNF is expected.

### DISCLOSURE OF THE INVENTION

Against the background of the prior art described above, the problems to be solved by the present invention is to eradicate the production of MMP, which is the main enzyme causing the joint destruction in the diseases like rheumatism. Namely, the object of the present invention is to provide the compounds inhibiting the production of MMP itself, and then to provide the compounds inhibiting the production of inflammatory cytokines such as TNF, which is one of the inducing factors of MMP.

The inventors investigated the compounds which can inhibit the production of MMP from human joint synovial cells and can inhibit the production of TNF from mouse peritoneal cells in order to overcome the said problems. As a result, they have found the compounds of general formula (I) and completed the present invention.

Namely, the present invention provides polyazanaphthalene compounds of the following general formula (I) or pharmaceutically acceptable salts thereof:

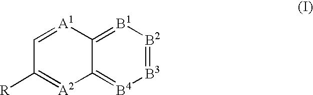

wherein A¹ and A² may be the same or different from each other and represent a nitrogen atom or —CH—, B¹ to B⁴ may be the same or different from each other and represent a nitrogen atom or —CR⁶—wherein R⁶ represents a hydrogen atom, halogen atom, an alkyl group, alkoxy group, alkylthio group and amino group, and the amino group may be substituted by one or two same or different alkyl group, alkenyl group, aryl group or amino-protecting group, R represents the following general formula (II):

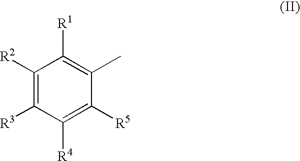

wherein R¹ to R⁵ may be the same or different from each other and represent a hydrogen atom, halogen atom, hydroxy group, mercapto group, nitro group, cyano group, trifluoromethyl group, an alkyl group, alkoxy group, alkylthio group, amino group, acyloxy group, acyl group, carboxyl group, alkoxycarbonyl group, or carbamoyl group, and at least one of R¹ to R⁵ represent(s) a group other than a hydrogen atom, and the amino group may be substituted by one or two same or different alkyl group, alkenyl group, aryl group or amino-protecting group,

or an aromatic heterocyclic group having one or more hetero atoms which may have a substituent(s). However, at least one of A¹ and A² represent a nitrogen atom, at least one of B¹ to B⁴ represent a nitrogen atom, and when A¹ and B¹ are a nitrogen atom and R¹ is either a hydroxy group or methoxy group, at least one of R² to R⁵ represent a group other than a hydrogen atom.

The present invention also provides an inhibitor of tissue degrading enzymes such as MMP and an inhibitor of production of inflammatory cytokines such as TNF, which contain the above-mentioned polyazanaphthalene compounds or pharmaceutically acceptable salts thereof The medicine can be used for the treatment of the various diseases associated with these enzymes and cytokines, for example, chronic rheumatoid arthritis, osteoarthritis, allergic diseases, psoriasis, transplant rejection, arterial sclerosis, ischemic re-perfusion disorder, diabetic nephritic and ocular diseases, cancer, autoimmune glomerulonephritis, infectious diseases, Crohn's disease, inflammatory intestinal diseases and autoimmune hepatitis. Especially, among the above-mentioned compounds, the polyazanaphthalene compounds wherein R² and R³ are an alkoxy group or pharmaceutically acceptable salts thereof produce a remarkable effect.

### BEST MODE FOR CARRYING OUT THE INVENTION

The detailed description of the present invention will be made below.

The compounds of the present invention are shown in the above-mentioned general formula (I), and the term “halogen atom” includes fluorine atom, chlorine atom, bromine atom and iodine atom. Among them, chlorine atom and bromine atom are preferred.

The term “alkyl group” indicates linear or branched alkyl groups having 1 to 6 carbon atoms such as methyl group, ethyl group, n-propyl group, isopropyl group, n-butyl group, isobutyl group, sec-butyl group, tert-butyl group, n-pentyl group, isopentyl group, tert-pentyl group, neopentyl group, 2-pentyl group, 3-pentyl group, n-hexyl group and 2-hexyl group. Among them, methyl group and ethyl group are preferred.

The term “alkoxy group” indicates linear or branched alkoxy groups having 1 to 6 carbon atoms which may be substituted; alkoxy groups which have a cyclic alkyl chain having 3 to 6 carbon atoms, or alkoxy groups having cyclic carbon chains which may be fused. Examples of the alkoxy group include methoxy group, ethoxy group, n-propoxy group, isopropoxy group, n-butoxy group, isobutoxy group, sec-butoxy group, tert-butoxy group, benzyloxy group, 2-phenylethoxy group, 3-phenylpropyloxy group, 4-phenylbutoxy group, 5-phenylpentyloxy group, 6-phenylhexyloxy group, cyclopropyloxy group, cyclobutoxy group, cyclopentyloxy group, cyclohexyloxy group, 1-indanyloxy group and 2-indanyloxy group. Among them, linear alkoxy group having 1 to 6 carbon atoms is preferred, linear alkoxy group having 1 or 2 carbon atoms is more preferred, and methoxy group is particularly preferred.

The term “alkylthio groups” indicates linear or branched alkylthio groups having 1 to 6 carbon atoms which may be substituted, such as methylthio group, ethylthio group, n-propylthio group, isopropylthio group, n-butylthio group, isobutylthio group, sec-butylthio group and tert-butylthio group. Among them, linear alkylthio group having 1 to 6 carbon atoms is preferred and methylthio group is particularly preferred.

The term “amino group which may be substituted with either one or two same or different alkyl groups, alkenyl groups, aryl groups or amino-protecting groups” indicates amino-groups which may have, on nitrogen atom of the amino group, one or two same or different alkyl groups having 1 to 6 carbon atoms, alkenyl groups having 1 to 6 carbon atoms; aryl groups having 5 to 10 atoms or amino groups which may have an amino-protecting group. Examples of the amino group include methylamino group, ethylamino group, propylamino group, isopropylamino group, arylamino group, butylamino group, phenylamino group, naphthylamino group, dimethylamino group, diethylamino group, dipropylamino group, diisopropylamino group, methylethylamino group, pyrrolidyl group and piperidyl group. Amino-protecting groups are usually used in organic synthesis and not particularly limited so far as they are capable of protecting the amino group from reactions. They include, for example, acyl groups such as formyl group, acetyl group and pivaloyl group; and alkoxycarbonyl groups such as methoxycarbonyl group, ethoxycarbonyl group, tert-butoxycarbonyl group and fluorenyl-9-methoxycarbonyl group. Among them, unsubstituted amino group, amino group substituted with one alkenyl group having 1 to 6 carbon atoms, and amino group substituted with one amino-protecting group are preferable.

The term “acyloxy group” indicates linear or branched acyloxy groups having 1 to 6 carbon atoms or acyloxy groups having a substituted or unsubstituted aryl group, such as formyloxy group, acetyloxy group, propionyloxy group, butyroyloxy group, isobutyroyloxy group, valeroyloxy group, isovaleroyloxy group, pivaloyloxy group, hexanoyloxy group, acryloyloxy group, methacryloyloxy group, crotonoyloxy group, isocrotonoyloxy group, benzoyloxy group and naphthoyloxy group. Among them, acetyloxy group, pivaloyloxy group and benzoyloxy group are preferred.

The term “acyl groups” indicates linear or branched acyl groups having 1 to 6 carbon atoms or acyl groups having a substituted or unsubstituted aryl group, such as formyl group, acetyl group, propionyl group, butyroyl group, isobutyroyl group, valeroyl group, isovaleroyl group, pivaloyl group, hexanoyl group, acryloyl group, methacryloyl group, crotonoyl group, isocrotonoyl group, benzoyl group and naphthoyl group. Among them, acetyl group, pivaloyl group and benzoyl group are preferred.

The term “alkoxycarbonyl groups” indicates alkoxy carbonyl groups whose alkoxy part has 1 to 6 carbon atoms such as methoxycarbonyl group, ethoxycarbonyl group, propoxycarbonyl group, isopropoxycarbonyl group, n-butoxycarbonyl group, isobutoxycarbonyl group, sec-butoxycarbonyl group and tert-butoxycarbonyl group. Among them, alkoxycarbonyl group whose alkoxy part has 1 to 3 carbon atoms is preferred.

The term “carbamoyl groups” indicates carbamoyl groups which may have one or two alkyl groups having 1 to 6 carbon atoms on a nitrogen atom. Examples of the carbamoyl group include carbamoyl group, N-methylcarbamoyl group, N-ethylcarbamoyl group, N,N-dimethylcarbamoyl group, N,N-diethylcarbamoyl group. Among them, carbamoyl group and N,N-dimethylcarbamoyl group are preferred.

The term “aromatic heterocyclic group having one or more hetero atoms” indicates 5-to-7-membered aromatic heterocyclic group constituted by a carbon atom, nitrogen atom, oxygen atom, sulfur atom, selenium and the like. The examples are pyridine, dihydropyran, pyridazine, pyrimidine, pyrazine, triazine, tetrazine, pyrrole, furan, thiophene, (oxazole), isooxazole, thiazole, isothiazole, imidazole, triazole, pyrazole, furazane and thiadiazole. Among them, pyridine and thiophene are preferred.

The term “which may have a substituent(s)” in the expression “aromatic heterocyclic groups having one or more hetero atoms, which may have a substituent(s)” indicates that these groups may have 1 to 5 substituents on the ring. The said substituents may be the same or different from each other and the position of the substituent is not particularly limited. The substituents are, for example, halogen atoms, alkyl groups and alkoxy group. Among them, methoxy group is preferred.

In general formula (I), it is preferred that A¹ and A² are different from each other. Two or three in B¹ to B⁴ are preferably —CR⁶—. It is particularly preferred that three in B¹ to B⁴ are —CR⁶. In this case, R⁶ is preferably a hydrogen atom, halogen atom (more preferably a chlorine atom), an amino group (more preferably unsubstituted amino group, amino group substituted with one alkenyl group having 1 to 6 carbon atoms, amino group substituted with one amino-protecting group.)

R¹ to R⁵ are preferably either a hydrogen atom, an alkyl group, alkoxy group, alkoxycarbonyl group, a halogen atom or thioalkyl group. Among them, a hydrogen atom or alkoxy group is preferred. Particularly it is preferred that three in R¹ to R⁵ are a hydrogen atom and other two are either an alkyl group, alkoxy group, alkoxycarbonyl group, a halogen atom or thioalkyl group, especially a hydrogen atom or an alkoxy group. Further, two in R¹ to R⁵ are preferably linear alkoxy group having 1 to 6 carbon atoms. It is most preferable that two in R¹ to R⁵ are unsubstituted linear alkoxy group having 1 to 6 carbon atoms.

The compounds have high activity when R in general formula (I) of the present invention is a group represented by general formula (II). The compounds also have high activity when A¹ is a nitrogen atom and A² is —CH—, or general formula (I) is either the following formula (III), (V), (V), (VI), or (VII), especially (III), (IV) or (V):

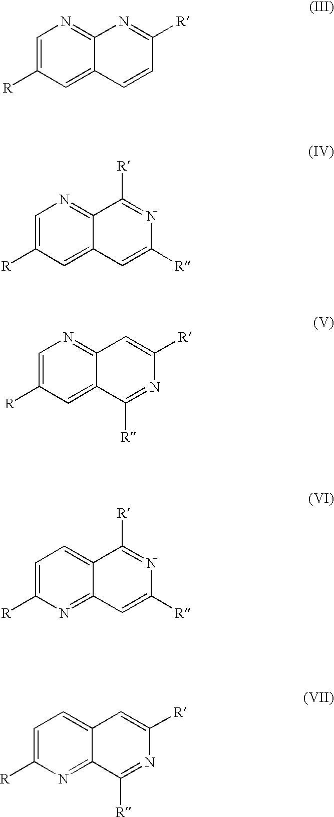

wherein R′ and R″ may be the same or different from each other and represent either a hydrogen atom, halogen atom, an alkyl group, alkoxy group, alkylthio group or amino group and the amino group may be substituted with one or two same or different alkyl groups, alkenyl groups, aryl groups or amino-protecting groups.

It is preferred that R is a group represented by general formula (II), A¹ is a nitrogen atom and A² is —CH—. It is also preferred that R is a group represented by general formula (II) and general formula (I) is the above-mentioned formulae (III), (IV), (V), (VI), or (VII) (especially (III), (IV) or (V)).

The compounds have higher activity when R² and R³ are an alkoxy group and R¹, R⁴ and R⁵ are a hydrogen atom in general formula (II).

The pharmaceutically acceptable salts are as follows: When the compounds of the present invention are completely acidic, the salts of them are, for example, ammonium salts, alkali metal salts (such as, preferably, sodium and potassium salts), alkaline earth metal salts (such as, preferably, calcium and magnesium salts) and organic base salts such as dicyclohexylamine salts, benzathine salts, N-methyl-D-glucan salts, hydramine salts, and salts of amino acids such as arginine and lysine. When the compounds of the present invention are completely alkaline, the salts of them are, for example, acid-addition salts such as those with inorganic acids, or example, hydrochloric acid, sulfuric acid, nitric acid and phosphoric acid, and also those with organic acids, for example, acetic acid, lactic acid, citric acid, tartaric acid, maleic acid, fumaric acid and monomethylsulfuric acid. If necessary, the salts may be in a hydrous form or in the form of hydrates.

The compounds of the present invention may be in the form of isomers such optical isomers and geometrical isomers, hydrates, solvated products and crystals of any form.

As for the naphthylidine compounds substituted with an aryl group which have a similar skeleton to the compounds of the present invention, those like the following compounds (VIII) and (IX) are known as having antibacterial activity (Yakugaku Zasshi, 99(5), 451-457 (1997)):

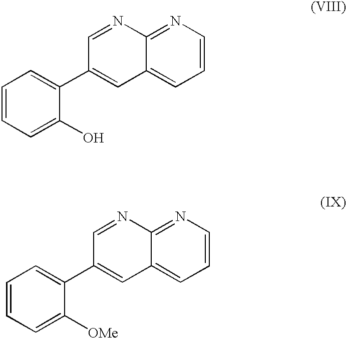

However, they do not suggest the activity inhibiting the production of MMP and inflammatory cytokines such as TNF described in the present invention. Methods for synthesizing the naphthylidine compounds having an unsubstituted aryl group represented by the following compounds (X) and (XI) are well known (Journal of Heterocyclic Chemistry, 11, 151 (1974), and 13, 387 (1996)):

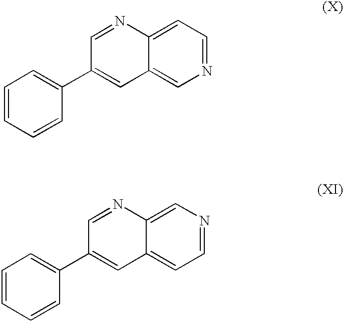

However, there is no description on bioactivity and, therefore, they do not suggest the activity inhibiting the production of MMP and inflammatory cytokines such as TNF described in the present invention.

Methods for producing the compounds of the present invention will be described below.

The compounds of the present invention can be produced by the conventional method. The common methods for production will be described below as reference.

For example, when R is a substituted benzene ring in the compound (I) of the present invention, the object compounds can be obtained by Suzuki Reaction of synthesized borate compounds corresponding to R with accommodating heterocyclic halide:

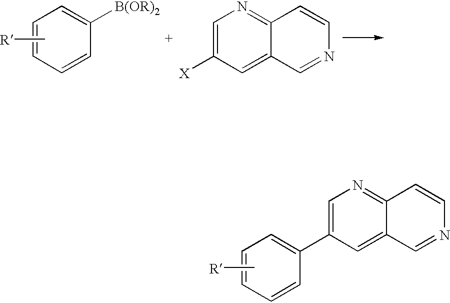

wherein R′ represents a substituent on a benzene ring, B(OR)₂ represents boronic acid such as boronic acid, catechol boronate and pinacol boronate or boronic ester, X represents a halogen atom such as bromine and iodine.

The compounds can be also produced by the same condensation forming reaction even if a borate substituent and halogen atom are reverse each other.

Other than the above methods, the compounds can be produced by the known aromatic ring-aromatic ring condensation forming reaction.

The above-mentioned compounds can be also produced by condensing aryl acetaldehyde compounds with amino aldehyde compounds in the presence of base as shown below:

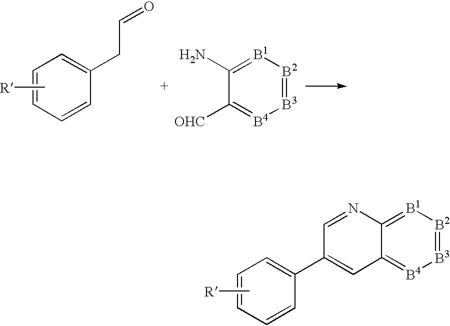

wherein R′ represents a substituent on a benzene ring, B¹ to B⁴ may be the same or different from each other and represent a nitrogen atom or C—R″ wherein R″ represents such as hydrogen atom, halogen atom, an alkoxy group, alkylthio group and amino group which may be substituted.

Further, the compounds that the position of a nitrogen atom is different from the above-mentioned compounds can be produced by condensing acetophenone compounds with amino aldehyde compounds in the presence of base as shown below:

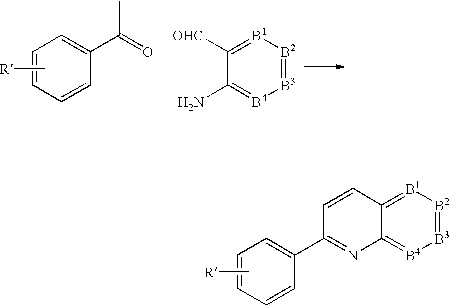

wherein R′ represents a substituent on a benzene ring, B¹ to B⁴ may be the same or different from each other and represent a nitrogen atom or C—R″ wherein R″ represents such as hydrogen atom, halogen atom, an alkoxy group, alkylthio group and amino group which may be substituted.

Further, the following compound can be produced by condensing α-keto aldehyde compounds with diamine compounds:

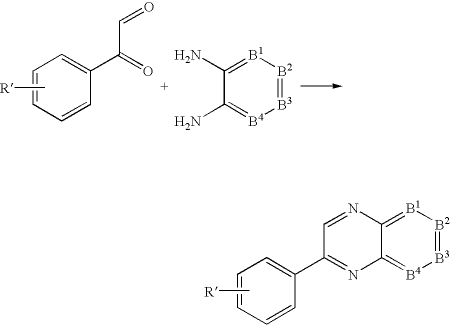

wherein R′ represents a substituent on a benzene ring, B¹ to B⁴ may be the same or different from each other and represent a nitrogen atom or C—R″ wherein R″ represents such as hydrogen atom, halogen atom, an alkoxy group, alkylthio group and amino group which may be substituted.

The compounds of the present invention can be also synthesized by applying these processes or by an ordinary process.

The compounds of the present invention obtained by the above-mentioned processes can be purified by a purification method usually employed in the synthesis of organic compounds such as extraction, distillation, crystallization or column chromatography.

The compounds of the present invention thus obtained can be used as the medicine useful for the treatment of various diseases associated with tissue degrading enzymes such as MMP and inflammatory cytokines such as TNF. Namely, they are useful for the treatment of, for example, chronic rheumatoid arthritis, osteoarthritis, allergic diseases, psoriasis, transplant rejection, arterial sclerosis, ischemic re-perfusion disorder, diabetic nephritic and ocular diseases, cancer, autoimmune glomerulonephritis, infectious diseases, Crohn's disease, inflammatory intestinal diseases and autoimmune hepatitis.

When the compounds of the present invention are used as anti-inflammatory agents, they can be given by oral, intravenous or percutaneous administration or by eye dropping method. The dosage, which varies depending on the symptoms and age of the patient and also administration method, is usually 1 to 3,000 mg/kg/day.

Preparations containing the compounds of the present invention can be prepared by an ordinary method. The preparations may be in the form of an injection, tablets, granules, grains, powders, capsules, cream or suppositories. Carriers used for producing the preparations are, for example, lactose, glucose, D-mannitol, starch, crystalline cellulose, calcium carbonate, kaolin, starch, gelatin, hydroxypropylcellulose, hydroxypropylmethylcellulose, polyvinylpyrrolidone, ethanol, carboxymethylcellulose, calcium salt of carboxymethylcellulose, magnesium stearate, talc, acetylcellulose, sucrose, titanium oxide, benzoic acid, parahydroxybenzoic esters, sodium dehydroacetate, gum arabic, tragacanth, methylcellulose, egg yolk, surfactants, simple syrup, citric acid, distilled water, ethanol, glycerol, propylene glycol, macrogol, sodium monohydrogenphosphate, sodium dihydrogenphosphate, sodium phosphate, glucose, sodium chloride, phenol, thimerosal, p-hydroxybenzoic esters and sodium hydrogensulfite. They are suitably selected depending on the form of the preparation, and mixed with the compounds of the present invention.

The amount of the active ingredient of the present invention in the preparation of the present invention is not particularly limited because it varies in a wide range depending on the form of the preparation. However, the amount is usually 0.01 to 100% by weight, and preferably 1 to 100% by weight, based on the whole composition.

### EXAMPLES

The following Examples will further illustrate the present invention, which by no means limit the invention.

Compounds synthesized in Examples 1 to 27 are as follows:

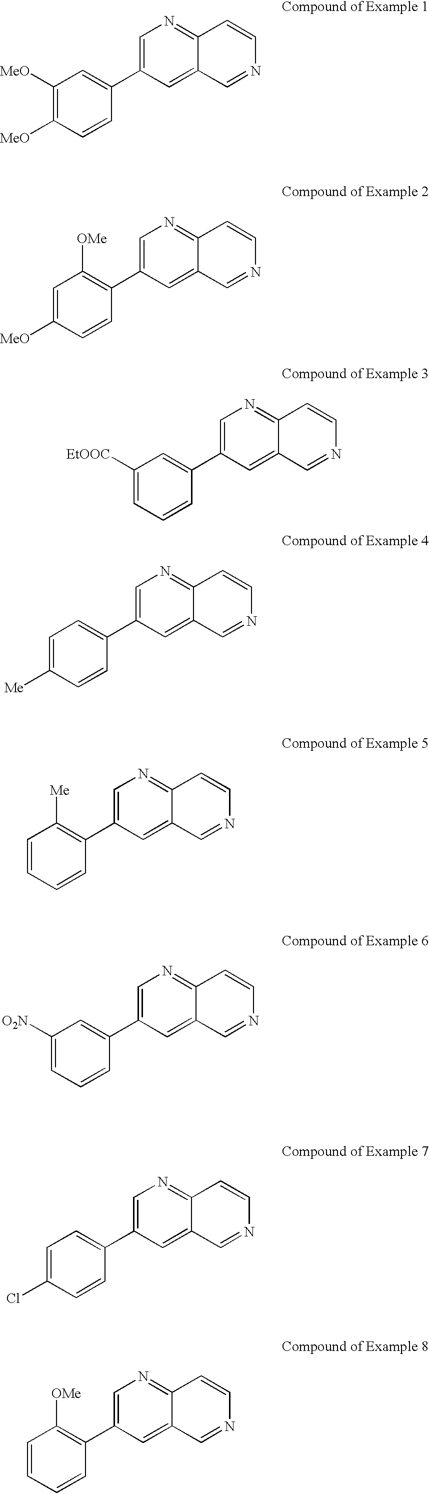

### Example 1

### Process for Producing 3-(3,4-Dimethoxyphenyl)-1,6-Naphtylidine

4-amino-3-pyridinecarboxyaldehyde (153.7 mg, 1.26 mmol) was dissolved in methanol (3 ml). A solution of 28%-sodium methoxide-methanol (1 ml) and then a solution of 3,4-dimethoxyphenylacetaldehyde (338.4 mg, 1.88 mmol) in methanol (2 ml) were added thereto and they were stirred at room temperature for 13 hours. After concentrating the reaction mixture under reduced pressure and adding water, the mixture was extracted with ethyl acetate twice, and then dried over anhydrous sodium sulfate and concentrated under reduced pressure. The obtained product was purified by silica gel column chromatography (dichloromethane/methanol) to obtain example compound 1 in the form of white crystals (79 mg, 23%).

¹H—NMR (300 MHz,CDCl₃) δ=3.97 (3H, s), 4.01 (3H, s), 7.05 (1H, d, J=8.1 Hz), 7.21 (1H, d, J=2.1 Hz), 7.29 (1H, d, J=2.1, 8.1 Hz), 7.95 (1H, m), 8.37 (1H, dd, J=0.9, 2.4 Hz), 8.76 (1H, d, J=5.7 Hz), 9.34-9.36 (2H, m).

### Example 2

### Process for Producing 3-(2,4-Dimethoxyphenyl)-1,6-Naphthylidine

[1,1-bis(diphenylphosphino)ferrocene] dichloropalladium(II) dichloromethane complex (1:1) (12.3 mg, 15 μmol), 1,1-bis(diphenylphosphino)ferrocene (8.3 mg, 15 μmol), bis(pinacolate)diboron (140.0 mg, 0.55 mmol), 1-bromo-2,4-dimethoxybenzene (108.5 mg, 0.50 mmol) and potassium acetate (147.0 mg, 1.5 mmol) were dissolved in toluene (2 ml) and stirred under argon atmosphere at 80° C. overnight. 3-Bromo-1,6-naphthylidine (52.3 mg, 0.25 mmol), [1,1-bis(diphenylphosphino)ferrocene] dichloropalladium(II) dichloromethane complex (1:1) (12.3 mg, 15 μmol), water solution of 2.5M-sodium carbonate (1 ml) and dimethylformamide (0.5 ml) were added thereto and stirred at 80° C. overnight. After the reaction was completed, water was added to the reaction mixture and the mixture was extracted with ethyl acetate. The obtained product was purified by silica gel column chromatography (hexane/ethyl acetate) to obtain example compound 2 in the form of pale brown power (12.5 mg, 19%).

¹H—NMR (300 MHz,CDCl₃) δ=3.85 (3H, s), 3.89 (3H, s), 6.63 (1H, d, J=2.3 Hz), 6.66 (1H, dd, J=2.3, 8.2 Hz), 7.36 (1H, d, J=8.2 Hz), 7.93 (1H, d, J=6.0 Hz), 8.34 (1H, dd, J=0.9, 2.4 Hz), 8.74 (1H, d, J=6.0 Hz). MS (ESI) m/z 267 (MH)⁺.

### Example 3

### Process for Producing 3-(3-Ethoxycarbonylphenyl)-1,6-Naphthylidine

Example compound 3 was obtained (52.4 mg, 75%) from 3-ethyl bromobenzoate (114.5 mg, 0.5 mmol) in the same manner as that in Example 2.

¹H—NMR (300 MHz,CDCl₃) δ=1.45 (3H, t, J=7.2 Hz), 4.45 (2H, q, J=7.2 Hz), 7.64 (1H, t, J=7.6 Hz), 7.90-7.93 (1H, m), 7.98 (1H, d, J=5.9 Hz), 8.16 (1H, dt, J=1.5, 7.6 Hz), 8.41 (1H, d, J=2.4 Hz), 8.49 (1H, d, J=2.4 Hz), 8.80 (1H, d, J=5.9 Hz), 9.38-9.39 (2H, m). MS (ESI) m/z 279 (MH)⁺.

### Example 4

### Process for Producing 3-(4-Methylphenyl)-1,6-Naphthylidine

3-Bromo-1,6-naphthylidine (30 mg, 0.14 mmol), 4-methylphenylboronic acid (29 mg, 0.22 mmol), sodium carbonate (61 mg, 0.57 mmol) and tetrakis(triphenylphosphine) palladium (8.3 mg, 7.2 μmol) were dissolved in the mixed solution of water (1 ml),ethanol (1 ml) and toluene (1 ml), and they were stirred under argon atmosphere at 60° C. for 2 hours. After cooling the reaction mixture and extracting with ethyl acetate, the obtained product was purified by silica gel column chromatography (hexane/ethyl acetate) to obtain example compound 4 in the form of pale yellow crystals (28.4 mg, 92%).

H—NMR (300 MHz,CDCl₃) δ=2.45 (3H, s), 7.36 (2H, d, J=8.1 Hz), 7.63 (2H, d, J=8.1 Hz), 7.95 (1H, d, J=5.7 Hz), 8.40 (1H, dd, J=0.9, 2.4 Hz), 8.76 (1H, d, J=5.7 Hz), 9.34 (1H, s), 9.36 (1H, d, J=2.4 Hz). MS (ESI) m/z 221 (MH)⁺.

### Example 5

### Process for Producing 3-(2-Methylphenyl)-1,6-Naphthylidine

Example compound 5 was obtained in the form of yellow oily product (30 mg, 95%) from 2-methylphenylboronic acid (29.3 mg, 0.22 mmol) in the same manner as that in Example 4.

¹H—NMR (300 MHz,CDCl₃) δ=2.34 (3H, s), 7.32-7.39 (4H, m), 7.98 (1H, d, J=6.2 Hz), 8.23 (1H, dd, J=0.9, 2.4 Hz), 8.80 (1H, d, J=6.2 Hz), 9.11 (1H, d, J=2.4 Hz), 9.33 (1H, s). MS (ESI) m/z 221 (MH)⁺.

### Example 6

### Process for Producing 3-(3-Nitrophenyl)-1,6-Naphthylidine

Example compound 6 was obtained in the form of white powder (1.4 mg, 4%) from 3-nitrophenylboronic acid (35.9 mg, 0.22 mmol) in the same manner as that in Example 4.

¹H—NMR (300 MHz,CDCl₃) δ=7.77 (1H, t, J=8.2 Hz), 8.01 (1H, d, J=6.0 Hz), 8.07 (1H, ddd, J=0.9, 1.9, 8.2 Hz), 8.35 (1H, ddd, J=0.9, 1.9, 8.2 Hz), 8.52 (1H, dd, J=0.9, 2.4 Hz), 8.61 (1H, t, J=1.9 Hz), 8.84 (1H, d, J=5.7 Hz), 9.39 (1H, d, J=2.4 Hz), 9.41 (1H, s). MS (ESI) m/z 252 (MH)⁺.

### Example 7

### Process for Producing 3-(4-Chlorophenyl)-1,6-Naphthylidine

Example compound 7 was obtained in the form of pale yellow powder (30.6 mg, 89%) from 4-chlorophenylboronic acid (33.7 mg, 0.22 mmol) in the same manner as that in Example 4.

¹H—NMR (300 MHz,CDCl₃) δ=7.52-7.55 (2H, m), 7.65-7.70 (2H, m), 7.97 (1H, d, J=6.0 Hz), 8.41 (1H, d, J=2.3 Hz), 8.79 (1H, d, J=6.0 Hz), 9.33 (1H, d, J=2.3 Hz), 9.36 (1H, s). MS (ESI) m/z 241 (MH)⁺.

### Example 8

### Process for Producing 3-(2-Methoxyphenyl)-1,6-Naphthylidine, Which is Not Included in the Claims of the Present Application

Example compound 8 was obtained in the form of pale yellow powder (28.3 mg, 83%) from 2-methoxyphenylboronic acid (32.7 mg, 0.22 mmol) in the same manner as that in Example 4.

¹H—NMR (300 MHz,CDCl₃) δ=3.87 (3H, s), 7.05-7.26 (2H, m), 7.42-7.47 (2H, m), 7.95 (1H, d, J=5.9 Hz), 8.39 (1H, dd, J=0.9,2.3 Hz), 8.76 (1H, d, J=5.9 Hz), 9.29 (1H, d, J=2.3 Hz), 9.31 (1H, d, J=0.9 Hz). MS (ESI) m/z 237 (MH)⁺.

### Example 9

### Process for Producing 3-(3-Methoxyphenyl)-1,6-Naphthylidine

Example compound 9 was obtained in the form of pale yellow powder (20.8 mg, 61%) from 3-methoxyphenylboronic acid (32.7 mg, 0.22 mmol) in the same manner as that in Example 4.

¹H—NMR (300 MHz,CDCl₃) δ=3.92 (3H, s), 7.02 (1H, ddd, J=0.9, 2.1, 7.9 Hz), 7.23 (1H, t, J=2.1 Hz), 7.30 (1H, ddd, J=0.9, 2.1, 7.9 Hz), 7.48 (1H, t, J=7.9 Hz), 7.97 (1H, d, J=6.0 Hz), 8.42 (1H, dd, J=0.9, 2.1 Hz), 8.78 (1H, d, J=6.0 Hz), 9.35-9.36 (2H, m). MS (ESI) m/z 237 (MH)+.

### Example 10

### Process for Producing 3-(4-Methoxyphenyl)-1,6-Naphthylidine

Example compound 10 was obtained in the form of pale yellow powder (15.6 mg, 46%) from 4-methoxyphenylboronic acid (32.7 mg, 0.22 mmol) in the same manner as that in Example 4.

¹H—NMR (300 MHz,CDCl₃) δ=3.91 (3H, s), 7.07-7.10 (2H, m), 7.66-7.68 (2H, m), 7.95 (1H, d, J=5.9 Hz), 8.37 (1H, d, J=2.4 Hz), 8.75 (1H, d, J=5.9 Hz), 9.33 (1H, s), 9.35 (1H, d, J=2.4 Hz). MS (ESI) m/z 237 (MH)⁺.

### Example 11

### Process for Producing 3-(3-Ethoxyphenyl)-1,6-Naphthylidine

Example compound 11 was obtained in the form of pale yellow powder (34.2 mg, 95%) from 3-ethoxyphenylboronic acid (35.7 mg, 0.22 mmol) in the same manner as that in Example 4.

¹H—NMR (300 MHz,CDCl₃) δ=1.48 (3H, t, J=7.0 Hz), 4.14 (2H, q, J=7.0 Hz), 7.01 (1H, ddd, J=0.9, 2.6, 8.1 Hz), 7.23-7.30 (2H, m), 7.46 (1H, t, J=8.1 Hz), 7.96 (1H, d, J=5.9 Hz), 8.42 (1H, dd, J=0.8, 2.3 Hz), 8.77 (1H, d, J=5.9 Hz), 9.35 (1H, d, J=0.8 Hz), 9.36 (1H, d, J=2.3 Hz). MS (ESI) m/z 251 (MH)⁺.

### Example 12

### Process for Producing 3-(4-Methylthiophenyl)-1,6-Naphthylidine

Example compound 12 was obtained in the form of pale yellow powder (17.6 mg, 49%) from 4-(methylthio) phenylboronic acid (36.2 mg, 0.22 mmol) in the same manner as that in Example 4.

¹H—NMR (300 MHz,CDCl₃) δ=2.56 (3H, s), 7.41-7.44 (2H, m), 7.64-7.68 (2H, m), 7.95 (1H, d, J=5.9 Hz), 8.40 (1H, dd, J=0.9, 2.4 Hz), 8.77 (1H, d, J=5.9 Hz), 9.34-9.35 (2H, m). MS (ESI) m/z 253 (MH)⁺.

### Example 13

### Process for Producing 3-(3-Trifluoromethyl)-1,6-Naphthylidine

Example compound 13 was obtained in the form of pale yellow powder (35.2 mg, 89%) from 3-trifluoromethylbenzene boronic acid (40.9 mg, 0.22 mmol) in the same manner as that in Example 4.

¹H—NMR (300 MHz,CDCl₃) δ=7.68-7.78 (2H, m), 7.91-8.01 (3H, m), 8.47-8.48 (1H, m), 8.83 (1H, d, J=5.7 Hz), 9.37 (1H, d, J=2.4 Hz), 9.40 (1H, s). MS (ESI) m/z 275 (MH)⁺.

### Example 14

### Process for Producing 3-(4-Trifluoromethoxyphenyl)-1,6-Naphthylidine

Example compound 14 was obtained in the form of pale yellow powder (35.5 mg, 85%) from 4-(trifluoromethoxy) benzene boronic acid (44.3 mg, 0.22 mmol) in the same manner as that in Example 4.

¹H—NMR (300 MHz,CDCl₃) δ=7.41-7.44 (2H, m), 7.75-7.78 (2H, m), 7.98-8.00 (1H, m), 8.43 (1H, dd, J=0.9, 2.4 Hz), 8.82 (1H, d, J=6.0 Hz), 9.35 (1H, d, J=2.4 Hz), 9.38 (1H, d, J=0.9 Hz). MS (ESI) m/z 291 (MH)⁺.

### Example 15

### Process for Producing 3-(3-Thienyl)-1,6-Naphthylidine

Example compound 15 was obtained in the form of pale yellow powder (23.6 mg, 77%) from 3-thiophene boronic acid (27.5 mg, 0.22 mmol) in the same manner as that in Example 4.

¹H—NMR (300 MHz,CDCl₃) δ=7.53-7.54 (2H, m), 7.72 (1H, t, J=2.1 Hz), 7.93 (1H, d, J=5.9 Hz), 8.40 (1H, d, J=2.4 Hz), 8.75 (1H, d, J=5.9 Hz), 9.33 (1H, s), 9.39 (1H, d, J=2.4 Hz). MS (ESI) m/z 213 (MH)⁺.

### Example 16

### Process for Producing 3-(2-Thienyl)-1,6-Naphthylidine

Example compound 16 was obtained in the form of pale yellow powder (28.8 mg, 95%) from 2-thiophene boronic acid (27.5 mg, 0.22 mmol) in the same manner as that in Example 4.

¹H—NMR (300 MHz,CDCl₃) δ=7.20 (1H, dd, J=3.8, 5.0 Hz), 7.46 (1H, dd, J=1.2, 5.0 Hz), 7.55 (1H, dd, J=1.2, 3.8 Hz), 7.92 (1H, d, J=5.9 Hz), 8.39 (1H, dd, J=0.9, 2.4 Hz), 8.75 (1H, d, J=5.9 Hz), 9.31 (1H, d, J=0.9 Hz), 9.39 (1H, d, J=2.4 Hz). MS (ESI) m/z 213 (MH)⁺.

### Example 17

### Process for Producing [3-(3,4-Dimethoxyphenyl)-1,6-Naphthylidine-7-yl]-2,2-Dimethylpropionamide Hydrochloride

(Process 1) N-(4-Amino-5-Formyl-2-Pyridyl)-2,2-Dimethylpropionamide Triethylamine (0.03 ml) and pivaloyl chloride (0.025 ml) were added to a solution of 4,6-diamino-3-pyridinecarboxyaldehyde (24.9 mg, 0.18 mmol) in dichloromethane (3.5 ml), and they were stirred at room temperature for 5 minutes. Then, triethylamine (0.03 ml) and pivaloyl chloride (0.025 ml) were added to the mixture and stirred at room temperature for 10 minutes, and five drops of methanol were added thereto. The obtained product was purified by silica gel column chromatography (ethyl acetate/hexane) to obtain N-(4-amino-5-formyl-2-pyridyl)-2,2-dimethylpropionamide in the form of white solid substance (30.9 mg, 77%).

¹H—NMR (300 MHz,CDCl₃) δ=1.32 (9H, s), 1.64 (2H, s), 7.59 (1H, s), 7.99 (1H, brs), 8.27 (1H, s), 9.79 (1H, s).

(Process 2) [3-(3,4-Dimethoxyphenyl)-1,6-Naphthylidine-7-yl]-2,2-Dimethylpropionamide Hydrochloride

Example compound 17 was obtained as hydrochloride in the form of yellow powder (22.2 mg, 41%) from N-(4-amino-5-formyl-2-pyridyl)-2,2-dimethylpropionamide (30 mg, 0.14 mmol) obtained in Process 1 and 3,4-dimethoxyphenylacetaldehyde (34 mg, 0.62 mmol) in the same manner as that in Example 1.

¹H—NMR (300 MHz, DMSO-d₆) δ=1.38 (9H, s), 3.92 (3H, s), 3.98 (3H, s), 7.18 (1H, d, J=9.0 Hz), 7.46-7.50 (2H, m), 8.85 (1H, m), 9.31 (1H, dd, J=0.8, 2.3 Hz), 9.48 (1H, d, J=0.8 Hz), 9.58 (H, d, J=2.3 Hz)

### Example 18

### 7-Amino-3-(3,4-Dimethoxyphenyl)-1,6-Naphthylidine Hydrochloride

N-[3-(3,4-dimethoxyphenyl)-1,6-naphthylidine-7-yl]-2,2-dimethylpropionamide hydrochloride obtained in Example 17 (19.8 mg) was suspended in water (2 ml) and concentrated hydrochloric acid (0.4 ml) and stirred at 80° C. for 17 hours. After cooling the mixture and neutralizing in a solution of sodium hydroxide, the mixture was extracted with ethyl acetate, dried over anhydrous magnesium sulfate and evaporated under reduced pressure and then yellow powder was obtained. The obtained powder was dissolved in ethyl acetate (3 ml) and hydrochloric acid was added thereto. Example compound 18 was obtained as hydrochloride in the form of dark red powder (16.0 mg, 81%) by evaporating under reduced pressure.

¹H—NMR (300 MHz, DMSO-d₆) δ=3.83 (3H, s), 3.90 (3H, s), 6.87 (1H, s), 7.14 (1H, d, J=8.5 Hz), 7.43 (1H, dd, J=2.1, 8.5 Hz), 7.47 (1H, d, J=2.1 Hz), 9.16 (1H, br), 9.22 (1H, s), 9.35 (1H, d, J=2.4 Hz)

### Example 19

### Process for Producing 5-Chloro-3-(3,4-Dimethoxyphenyl)-1,6-Naphthylidine

(Process 1) N-(2-Chloro-4-Pyridyl)-2,2-Dimethylpropionamide

4-amino-2-chloropyridine (2.01 g, 15.7 mmol) and triethylamine (2.5 ml) were dissolved in dichloromethane (20 ml), then a solution of pivaloyl chloride (2.1 ml) in dichloromethane (3 ml) was added thereto and stirred at room temperature for 7 hours. After adding methanol (3 ml) and concentrating the mixture under reduced pressure, the obtained product was purified by silica gel column chromatography (ethyl acetate/hexane) to obtain N-(2-chloro-4-pyridyl)-2,2-dimethylpropionamide in the form of white solid substance (2.93 g, 88%).

¹H—NMR (300 MHz,CDCl₃) δ=1.32 (9H, s), 7.37 (1H, dd, J=1.9, 5.7 Hz), 7.43 (1H, brs), 7.66 (1H, d, J=1.9 Hz), 8.26 (1H, d, J=5.7 Hz)

(Process 2) N-(2-Chloro-3-Formyl-4-Pyridyl)-2,2-Dimethylpropionamide

N-Butylithium (a solution of 1.6M hexane) (12 ml, 19 mmol) was added at −78° C. to a solution of 2,2,6,6-tetramethylpiperidine (3.06 g, 21.6 mmol) in tetrahydrofuran. 30 minutes later, a solution of N-(2-chloro-4-pyridyl)-2,2-dimethylpropionamide (1.05 g, 4.96 mmol) obtained in Process 1 in tetrahydrofuran (5 ml) was added thereto. After stirring the mixture at the same temperature for 2.5 hours, N-formylpiperidine (2.4 ml, 22 mmol) was added and stirred for 1.5 hours and then 20 ml of water was added. After raising the temperature of the mixture up to room temperature, the mixture was extracted with ethyl acetate, dried over anhydrous sodium sulfate and concentrated under reduced pressure. The obtained product was purified by silica gel column chromatography (ethyl acetate/hexane) to obtain N-(2-chloro-3-formyl-4-pyridyl)-2,2-dimethylpropionamide in the form of white solid substance (414 mg, 35%).

¹H—NMR (300 MHz,CDCl₃) δ=1.36 (9H, s), 8.38 (1H, d, J=6.3 Hz), 8.68 (1H, d, J=6.3 Hz), 10.56 (1H, s), 11.97 (1H, br)

(Process 3) 4-Amino-2-Chloro-3-Pyridinecarboxyaldehyde

N-(2-chloro-3-formyl-4-pyridyl)-2,2-dimethylpropionamide (300 mg, 1.25 mmol) obtained in Process 2 was stirred in 2M hydrochloric acid (20 ml) at 90° C. for 22 hours. After lowering the temperature of the mixture to room temperature and neutralizing in sodium hydrogen carbonate, the mixture was extracted with ethyl acetate, dried over anhydrous sodium sulfate and concentrated under reduced pressure. Thus, 4-amino-2-chloro-3-pyridinecarboxyaldehyde was obtained in the form of white solid substance (147 mg, 76%).

¹H—NMR (300 MHz,CDCl₃) δ=4.8-5.6 (1H, br), 6.48 (1H, dd, J=0.5, 5.9 Hz), 7.97 (1H, d, J=5.9 Hz), 8.2-9.2 (1H, br), 10.44 (1H, d, J=0.5 Hz)

(Process 4) 5-Chloro-3-(3,4-Dimethoxyphenyl)-1,6-Naphthylidine

Example compound 19 was obtained in the form of white crystals (73.6 mg, 65%) from 4-amino-2-chloro-3-pyridinecarboxyaldehyde (59 mg, 0.376 mmol) and 3,4-dimethoxyphenylacetaldehyde (84 mg, 0.467 mmol) in the same manner as that in Example 1.

¹H—NMR (300 MHz,CDCl₃) δ=3.98 (3H, s), 4.02 (3H, s), 7.06 (1H, d, J=8.1 Hz), 7.22 (1H, d, J=2.2 Hz), 7.31 (1H, dd, J=2.2, 8.1 Hz), 7.90 (1H, dd, J=0.8, 6.0 Hz), 8.50 (1H, d, J=6.0 Hz), 8.67 (1H, dd, J=0.8, 2.2 Hz), 9.36 (1H, d, J=2.2 Hz)

### Example 20

### Process for Producing 5-Amino-3-(3,4-Dimethoxyphenyl)-1,6-Naphthylidine

A solution of 2M-ammonia-methanol (15 ml) as added to 5-chloro-3-(3,4-dimethoxyphenyl)-1,6-naphthylidine (34.8 mg, 0.116 mmol) which is example compound 19 and stirred in a sealed tube at 130° C. for 38 hours. After cooling and concentrating the mixture under reduced pressure, the obtained product was purified by silica gel column chromatography (ethyl acetate/hexane) and evaporated under reduced pressure to obtain example compound 20 in the form of golden yellow powder (17.9 mg, 55%).

¹H—NMR (300 MHz, DMSO-d₆) δ=3.83 (3H, s), 3.91 (3H, s), 6.99 (1H, d, J=6.0 Hz), 7.13 (1H, d, J=8.4 Hz), 7.18 (2H, brs), 7.43-7.47 (2H, m), 7.98 (1H, d, J=6.0 Hz), 8.85 (1H, br), 9.29 (1H, d, J=2.1 Hz)

### Example 21

### Process for Producing 5-Allylamino-3-(3,4-Dimethoxyphenyl)-1,6-Naphthylidine Hydrochloride

Allylamine (0.05 ml) was added to a solution of 5-chloro-3-(3,4-dimethoxyphenyl)-1,6-naphthylidine (30 mg, 0.1 mmol) which is example compound 19, [1,1-bis(diphenylphosphino)ferrocene] dichloropalladium(II) dichloromethane complex (1:1) (4.4 mg), 1,1-bis(diphenylphosphino)ferrocene (9.4 mg) in tetrahydrofuran (2 ml) and stirred under heating at 80° C. for 2 hours. After the reaction was completed, the mixture was concentrated under reduced pressure and purified by silica gel column chromatography (ethyl acetate). The obtained oily product was dissolved in ethyl acetate (3 ml) and hydrochloric acid was added thereto. The product was concentrated under reduced pressure to obtain example compound 21 in the form of orange color powder (12.2 mg, 40%).

¹H—NMR (300 MHz, DMSO-d₆) δ=3.85 (3H, s), 3.94 (3H, s), 4.30-4.40(2H, m), 5.26 (1H, d, J=10.5 Hz), 5.36 (1H, d, J=17.1 Hz), 6.00-6.10(1H, m), 7.16 (1H, d, J=8.7 Hz), 7.26 (1H, d, J=7.2 Hz), 7.56 (1H, dd, J=2.1, 8.1 Hz), 7.61(1H, d, J=1.8 Hz), 7.83(1H, d, J=7.2 Hz), 9.52(1H, s), 9.57(1H, d, J=1.8 Hz).

### Example 22

### Process for Producing 3-(3,4-Dimethoxyphenyl)-1,7-Naphthylidine

Example compound 22 was obtained (31.1 mg, 27%) from 3-amino-4-pyridinecarboxyaldehyde (53.4 mg, 0.437 mmol) and 3,4-dimethoxyphenylacetaldehyde (111.5 mg, 0.619 mmol) in the same manner as that in Example 1.

¹H—NMR (300 MHz,CDCl₃) δ=3.98 (3H, s), 4.01 (3H, s), 7.05 (1H, d, J=8.3 Hz), 7.22 (1H, d, J=2.1 Hz), 7.31 (1H, dd, J=2.1, 8.3 Hz), 7.70 (1H, dd, J=0.8, 5.8 Hz), 8.22 (1H, dd, J=0.8, 2.2 Hz), 8.64 (1H, d, J=5.8 Hz), 9.27 (1H, d, J=2.2 Hz), 9.54 (1H, d, J=0.8 Hz)

### Example 23

### Process for Producing 3-(3,4-Dimethoxyphenyl)-1,8-Naphthylidine

Example compound 23 was obtained in the form of white crystals (252 mg, 49%) from 2-amino-3-pyridinecarboxyaldehyde (239 mg, 1.96 mmol) and 3,4-dimethoxyphenylacetaldehyde (1.94 mmol) in the same manner as that in Example 1.

¹H—NMR (300 MHz,CDCl₃) δ=3.97 (3H, s), 4.00 (3H, s), 7.04 (1H, d, J=8.4 Hz), 7.22 (1H, d, J=2.1 Hz), 7.30 (1H, dd, J=2.1, 8.4 Hz), 7.52 (1H, dd, J=4.2, 8.2 Hz), 8.23-8.28 (2H, m), 9.12 (1H, dd, J=1.9 Hz), 9.39 (1H, d, J=2.5 Hz)

### Example 24

### Process for producing 2-(3,4-Dimethoxyphenyl)-1,6-Naphthylidine

4-amino-3-pyridinecarboxyaldehyde (50 mg, 0.41 mmol) and 3,4-dimethoxyacetophenone (88 mg, 0.49 mmol) were dissolved in methanol (2.5 ml) and stirred at room temperature for 12 hours after adding a solution of 28%-sodium methoxide-methanol (0.5 ml). The reaction mixture was concentrated under reduced pressure, extracted with ethyl acetate (80 ml) twice and dried over anhydrous sodium sulfate. The obtained product was concentrated under reduced pressure to obtain example compound 24 in the form of white solid substance (75.8 mg, 70%).

¹H—NMR (300 MHz,CDCl₃) δ=3.98 (3H, s), 4.06 (3H, s), 7.02 (1H, d, J=8.5 Hz), 7.72 (1H, dd, J=2.1 Hz), 7.91 (1H, d, J=2.1 Hz), 7.95 (1H, dd, J=0.8, 5.9 Hz), 7.99 (1H, d, J=8.7 Hz), 8.30 (1H, dd, J=0.8, 8.7 Hz), 8.75 (1H, d, J=5.9 Hz), 9.24 (1H, d, J=0.8 Hz)

### Example 25

### Process for Producing 2-(3,4-Dimethoxyphenyl)-1,7-Naphthylidine Hydrochloride

The reaction and after-treatment were done in the same manner as that in Example 23, by using 3-amino-4-pyridinecarboxyaldehyde (50.3 mg, 0.412 mmol) and 3,4-dimethoxyacetophenone (88 mg, 0.49 mmol) as raw materials. The obtained product was dissolved in ethyl acetate (2 ml) and evaporated under reduced pressure after adding hydrochloric acid to obtain example compound 25 in the form of dark red powder (60.4 mg, 42%)

¹H—NMR (300 MHz, DMSO-d₆) δ=3.88 (3H, s), 3.93 (3H, s), 7.19 (1H, d, J=9.0 Hz), 7.95-8.00 (2H, m), 8.32 (1H, d, J=6.0 Hz), 8.67 (1H, s), 8.70 (1H, d, J=6.0 Hz), 9.72 (1H, s)

### Example 26

### Process for Producing 3-(3-Cyclopentyloxy-4-Methoxyphenyl)-1,6-Naphthylidine

Example compound 26 was obtained in the form of yellow powder (120 mg, 75%) from 3-cyclopentyloxy-4-methoxybromobenzene (271 mg, 1 mmol) in the same manner as that in Example 2.

¹H—NMR (300 MHz,CDCl₃) δ=1.60-1.70 (2H, m), 1.85-2.05(6H, m), 3.93 (3H, s), 4.90-4.95 (1H, m), 7.03 (1H, d, J=8.7 Hz), 7.22 (1H, d, J=2.1 Hz), 7.27 (1H, d, J=2.1, 8.7 Hz), 7.94 (1H, d, J=6.0 Hz), 8.34 (1H, dd, J=0.9, 2.4 Hz), 8.76 (1H, d, J=6.0 Hz), 9.33-9.35 (2H, m). MS (ESI) m/z 321 (MH)⁺.

### Example 27

### Process for Producing 3-[3-(4-Phenylbutyloxy)-4-Methoxyphenyl]-1,6-Naphthylidine

Example compound 27 was obtained (160 mg, 84%) from 3-(4-phenylbutyloxy)-4-methoxybromobenzene (335 mg, 1 mmol) in the same manner as that in Example 2.

¹H—NMR (300 MHz,CDCl₃) δ=1.80-2.00 (4H, m), 2.72 (2H, t, J=7.2 Hz), 3.94 (3H, s), 4.14 (2H, t, J=7.2 Hz), 7.04 (1H, d, J=8.4 Hz), 7.15-7.30 (7H, m), 7.95 (1H, d, J=5.7 Hz), 8.34 (1H, dd, J=0.9, 2.4 Hz), 8.76 (1H, d, J=5.7 Hz), 9.32-9.34 (2H, m). MS (ESI) m/z 385 (MH)⁺.

### Example 28

### Evaluation of Inhibiting Production of Matrix Metalloprotease (MMP) Collected From Synovial Cells

The human joint normal synovial interstitial cells (cell System-SS cells, Dainippon Pharmaceutical Co., Ltd.) were seeded on a 96-well plate in a concentration of 1×10⁴ cells/well and cultured overnight. 30 minutes after the addition of the subject pharmaceutical compounds in a proper concentration, IL-1β was added to each well so as to obtain the final concentration of 0.5 ng/ml. After culturing overnight (16 to 18 hours) again, a culture fluid was collected and the amount of MMP-3 (stromelysin-1) in the culture fluid was determined. MMP-3 was determined with a test kit for determining stromelysin-1 (SL) in serum (Fuji Chemical Industries Ltd.), in accordance with an instruction book attached to the kit. When no stimulation was seen, the production amount was defined as 0% and when only IL-1β stimulation was seen, the amount was defined as 100%. The concentration was determined so that each subject pharmaceutical compound showed 50% inhibition.

The results of the evaluation are shown in Table 1.

TABLE 1

| Example No. | MMP Inhibition Activity (IC50: μg/ml) |
|---|---|
| 1 | 1.25 |
| 18 | 0.31 |
| 20 | 0.16 |
| 22 | 1.25 |
| 24 | 1.25 |

### Example 29

### Evaluation of Inhibiting Production of Tumor Necrosis Factors (TNF)

RPMI medium was injected into a peritoneal cavity of Balb/c mouse (Charles River Japan, Inc.) and the peritoneal cells were collected. The peritoneal cells were seeded on a 96-well plate in a concentration of 1×10⁴ cells/well. 30 minutes after the addition of the subject pharmaceutical compounds in a proper concentration, LPS (sigma, L-5886) was added to each well so as to obtain the final concentration of 10 μg/ml. After culturing overnight (16 to 18 hours) again, a culture fluid was collected and the amount of TNF α in the culture fluid was determined. Mouse TNF α was determined by the means of a sandwitch ELISA and anti-mouse TNF a hamster monoclonal antibody (Genzyme 1221-00) was used as the first antibody, anti-mouse TNF α rabbit polyclonal antibody (Genzyme, IP-400) as the second antibody, and alkaliphosphatase conjugate anti-rabbit IgG polyclonal goat antibody as the third antibody. Then, optical absorbance of alkaliphosphatase substrate (p-nitrophosphate) was determined and converted into the production amount. When no stimulation was seen, the production amount was defined as 0% and when only IL-1β stimulation was seen, the amount was defined as 100%. The concentration was determined so that each subject pharmaceutical compound showed 50% inhibition.

The results of the evaluation are shown in Table 2.

TABLE 2

| Example No. | TNF Inhibition Activity (IC50: μg/ml) |
|---|---|
| 1 | 0.5 |
| 18 | 0.6 |
| 19 | 1 |
| 20 | 0.3 |
| 22 | 0.02 |
| 23 | 0.1 |
| 24 | 0.6 |
| 26 | 0.01 |

It is obvious from the results shown above that the compounds of the present invention can be used as pharmaceutical compounds for the treatment of various diseases associated with tissue degrading enzymes such as MMP and inflammatory cytokines such as TNF. Namely, the subject compounds are useful as medicine for the treatment of diseases, for example, chronic rheumatoid arthritis, osteoarthritis, arterial sclerosis, diabetic nephritic and ocular diseases, cancer, autoimmune glomerulonephritis, infectious diseases, Crohn's disease, inflammatory intestinal diseases and autoimmune hepatitis.

What is claimed is:

1. The polyazanaphthalene compounds of the following general formula (1) or pharmaceutically acceptable salts thereof: 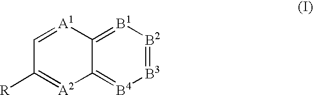
wherein A¹ and A² may be the same or different from each other and represent a nitrogen atom or —CH—, B¹ to B⁴ may be the same or different from each other and represent a nitrogen atom or —CR⁶—wherein R⁶ represents a hydrogen atom, halogen atom, an alkyl group, alkoxy group, alkylthio group and amino group, and the amino group may be substituted by one or two same or different alkyl group, alkenyl group, aryl group or amino-protecting group, and R represents the following general formula (II): 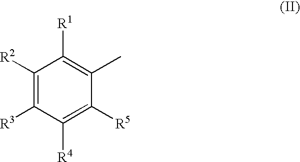
wherein R¹ to R⁵ may be the same or different from each other and represent a hydrogen atom, halogen atom, hydroxy group, mercapto group, nitro group, cyano group, trifluoromethyl group, an alkyl group, alkoxy group, alkylthio group, amino group, acyloxy group, acyl group, a carboxyl group, an alkoxycarbonyl group, or a carbamoyl group, and at least one of R¹ to R⁵ represent a group other than a hydrogen atom, and the amino group may be substituted by one or two same or different alkyl group, alkenyl group, aryl group or amino-protecting group,
or an aromatic heterocyclic group having one or more hetero atoms which may have a substituent(s), provided that at least one of A¹ and A² represent a nitrogen atom, at least one of B¹ to B⁴ represent a nitrogen atom, and when A¹ and B¹ are a nitrogen atom and R¹ is either a hydroxy group or methoxy group, at least one of R² to R⁵ represent a group other than a hydrogen atom.

2. The polyazanaphthalene compounds or pharmaceutically acceptable salts of claim 1, wherein R represents a group represented by the general formula (II).

3. The polyazanaphthalene compounds or pharmaceutically acceptable salts of claim 1, wherein R represents an aromatic heterocyclic group having one or more sulfur atoms.

4. The polyazanaphthalene compounds or pharmaceutically acceptable salts of claim 1, wherein two or three of B¹ to B⁴ represent —C R⁶—wherein R⁶ is defined in claim 1.

5. The polyazanaphthalene compounds or pharmaceutically acceptable salts of claim 1 or 2, wherein A¹ is a nitrogen atom and A² is —CH—.

6. The polyazanaphthalene compounds or pharmaceutically acceptable salts of claim 4, wherein R⁶ is either a hydrogen atom, halogen atom or an amino group wherein amino group is defined in claim 1.

7. The polyazanaphthalene compounds or pharmaceutically acceptable salts of claim 2, wherein the general formula (I) is either the following formula (III), (IV), (V), (VI), or (VII): 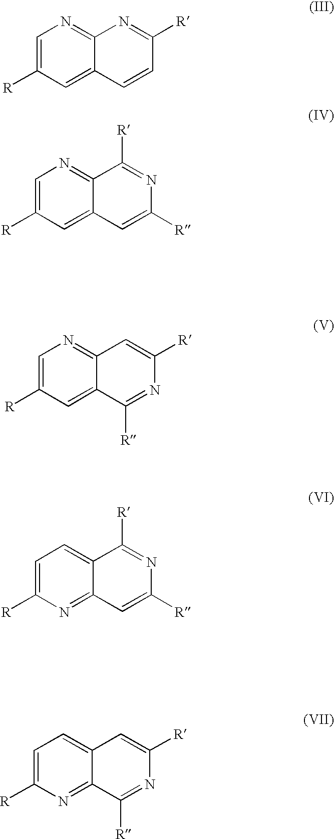
wherein R′ and R″ may be the same or different from each other and represent either a hydrogen atom, halogen atom, an alkyl group, alkoxy group, alkylthio group or amino group, provided that the amino group may be substituted with one or two same or different alkyl groups, alkenyl groups, aryl groups or amino-protecting groups.

8. The polyazanaphthalene compounds or pharmaceutically acceptable salts of claim 2, wherein the general formula (I) is either a formula (III), (IV) or (V) of claim 7.

9. The polyazanaphthalene compounds or pharmaceutically acceptable salts of claim 2, wherein R¹ to R⁵ represent a hydrogen atom, an alkyl group, alkoxy group, alkoxycarbonyl group, a halogen atom or thioalkyl group.

10. The polyazanaphthalene compounds or pharmaceutically acceptable salts of claim 9, wherein R¹ to R⁵ represent a hydrogen atom or an alkoxy group.

11. The polyazanaphthalene compounds or pharmaceutically acceptable salts of claim 10, wherein two of R¹ to R⁵ are an alkoxy group and other three are a hydrogen atom.

12. The polyazanaphthalene compounds or pharmaceutically acceptable salts of claim 11, wherein the alkoxy group represents a linear alkoxy group having 1 to 6 carbon atoms.

13. The polyazanaphthalene compounds or pharmaceutically acceptable salts of claim 5, wherein R² and R³ of the general formula (II) are an alkoxy group and R¹, R⁴ and R⁵ are a hydrogen atom.

14. The polyazanaphthalene compounds or pharmaceutically acceptable salts of claim 13, wherein R² and R³ are an alkoxy group having one or two carbon atoms.

15. The polyazanaphthalene compounds or pharmaceutically acceptable salts of claim 5, wherein R² and R³ of the general formula (II) are an alkoxy group and R¹, R⁴ and R⁵ are a hydrogen atom.

16. The polyazanaphthalene compounds or pharmaceutically acceptable salts of claim 15, wherein R² and R³ are an alkoxy group having one or two carbon atoms.

17. An inhibitor of matrix metalloprotease (MMP) production having the polyazanaphthalene compounds or pharmaceutically acceptable salts described in either claim 1 or 16 as active ingredients.

18. An inhibitor of tumor necrosis factor (TNF) production comprising the polyazanaphthalene compounds or pharmaceutically acceptable salts set out in any one of claim 1 or 16 as active ingredients.

19. A medicine comprising the polyazanaphthalene compounds or pharmaceutically acceptable salts set out in any one of claim 1 or 16 as active ingredients, for the treatment of either chronic rheumatoid arthritis, osteoarthritis, allergic diseases, psoriasis, transplant rejection, arterial sclerosis, ischemic re-perfusion disorder, diabetic nephritic and ocular diseases, cancer, autoimmune glomerulonephritis, infectious diseases, Crohn's disease, inflammatory intestinal diseases or autoimmune hepatitis.

20. A pharmaceutical compounds comprising the polyazanaphthalene compounds or pharmaceutically acceptable salts represented by the general formula (I) of any one of claims 1 to 16.
